(19) United States
(12) Patent Application Publication
(10) Pub. No.: US 2021/0317055 A1
(43) Pub. Date: Oct. 14, 2021

(54) PRODUCTION OF HALOOLEFINS IN AN ADIABATIC REACTION ZONE

(71) Applicant: THE CHEMOURS COMPANY FC, LLC, WILMINGTON, DE (US)

(72) Inventors: KARL R. KRAUSE, KENNETT SQUARE, PA (US); CONCETTA LA MARCA, WILMINGTON, DE (US); MARIO JOSEPH NAPPA, LEESBURG, FL (US); XUEHUI SUN, KENNETT SQUARE, PA (US)

(73) Assignee: THE CHEMOURS COMPANY FC, LLC, WILMINGTON, DE (US)

(21) Appl. No.: 17/261,043

(22) PCT Filed: Jul. 18, 2019
(86) PCT No.: PCT/US2019/042362

§ 371 Date: Jan. 18, 2021

Related U.S. Application Data
(60) Provisional application No. 62/699,968, filed on Jul. 18, 2018.

(51) Int. Cl. C07C 17/25; B01J 19/00; B01J 19/24

(52) U.S. Cl. CPC C07C 17/25; B01J 2219/00103; B01J 19/245; B01J 19/0013

(57) ABSTRACT

A process for producing at least one haloolefin by dehydrohalogenating a hydrohaloalkane. The dehydrohalogenation process is performed in the liquid phase or vapor phase in the presence or absence of a catalyst at a temperature sufficient to effect conversion of the hydrohaloalkane to a haloolefin (haloalkene) in an adiabatic reaction zone. In particular, the adiabatic reaction zone comprises at least two serially-connected adiabatic reactors and having a heat exchanger disposed in sequence and in fluid communication between each two reactors in series.

### FIELD

The present disclosure relates to a process to produce haloolefins, such as fluoropropenes, in an adiabatic reaction zone.

### BACKGROUND

Hydrochlorocarbons (HCCs), hydrochlorofluorocarbons (HCFCs) and chlorofluorocarbons (CFCs) are versatile compounds and have been employed in a wide range of applications, including their use as aerosol propellants, refrigerants, cleaning agents, expansion agents for thermoplastic and thermoset foams, heat transfer media, gaseous dielectrics, fire extinguishing and suppression agents, power cycle working fluids, polymerization media, particulate removal fluids, carrier fluids, buffing abrasive agents, and displacement drying agents. Industry has been working for the past few decades to find replacements for HCCs, HCFCs, and CFCs that have lower ozone depletion potential and other environmental benefits. In the search to replace HCCs, CFCs and HCFCs, many industries turned to the use of hydrofluorocarbons (HFCs).

HFCs do not contribute to the destruction of stratospheric ozone, but are of concern due to their contribution to the “greenhouse effect”, i.e., they contribute to global warming. As a result of their contribution to global warming, HFCs have come under scrutiny, and their widespread use may be limited in the future as has occurred for CFCs and HCFCs. Thus, there is a need for chemical compounds that have both low ozone depleting potentials (ODPs) and low global warming potentials (GWPs).

Certain hydrofluoroolefins (HFOs) have been identified as having both low ODPs and low GWPs. CF₃CF═CH₂ (HFO-1234yf) and CF₃CH═CHF (HFO-1234ze), both having zero ozone depletion and low global warming potential, have been identified as potential refrigerants. Other hydrofluoroolefins such as CF₃CH═CHCF₃ (HFO-1336mzz) and the hydro(fluoro)chloroolefins CF₃—CH═CHCl (HCFO-1233zd) have been identified as blowing agents. Other HFOs also have value as alternatives in other applications.

Hydrofluoroolefins and intermediates for producing hydrofluoroolefins may be produced by dehydrohalogenation of hydrochloroalkanes, hydrochlorofluoroalkanes or hydrofluoroalkanes, collectively, “hydrohaloalkanes”.

Chloroolefins, chlorofluoroolefins, and fluoroolefins, collectively, “haloolefins”, may all be desired products for example, for use as intermediates to produced desirable chemical compounds that have both low ozone depleting potentials (ODPs) and low global warming potentials (GWPs). For example, chloroolefins, chlorofluoroolefins and fluoroolefins may all be intermediates used to produce HFO-1234yf or HFO-1234ze or HFO-1336mzz, or HCFO-1233zd.

Dehydrohalogenation reactions generate corrosive HCl or HF. Dehydrohalogenation reactions can be catalytic or pyrolytic. Such reactions may performed at relatively high temperature (such as, for example greater than 180° C. for catalytic reactions or greater than 350° C. for pyrolytic reactions). Dehydrohalogenation reactions are also endothermic, and thus reaction rate is very sensitive to temperature/heat supply.

The aforementioned characteristics of a dehydrohalogenation reaction must be accommodated in process design and reaction zone. In a typical dehydrohalogenation process, a single multi-tubular reactor is used to facilitate heat transfer and maintain temperature of the endothermic reaction.

### SUMMARY

The present disclosure relates to a process for producing a product comprising at least one haloolefin (haloalkene) by dehydrohalogenating a hydrohaloalkane. The process is thus a dehydrohalogenation process. The process is performed in the liquid phase or in the vapor phase in the presence or absence of a catalyst at a temperature sufficient to effect conversion of the hydrohaloalkane to a haloolefin in an adiabatic reaction zone. In particular, the adiabatic reaction zone comprises at least two serially-connected adiabatic reactors having a heat exchanger disposed in sequence and in fluid communication between each two reactors in series. In other words, the reaction zone comprises at least two reactors, each reactor operating adiabatically, arranged in series, wherein a heat exchanger is arranged between two reactors in series. The process further comprises recovering a product comprising a haloolefin from the reaction zone.

Thus, according to one aspect of the present disclosure, there is provided a process for dehydrohalogenating a hydrohaloalkane in an adiabatic reaction zone, which process comprises the steps of:

(a) providing an adiabatic reaction zone comprising at least two serially-connected adiabatic reactors and having a heat exchanger disposed in sequence and in fluid communication between each two reactors in series;

(b) introducing a starting material comprising a hydrohaloalkane into a first adiabatic reactor of the serially-connected reactors, producing a reaction product;

(c) passing the reaction product from a preceding reactor to a heat exchanger, producing an intermediate product;

(d) introducing the intermediate product from the heat exchanger to a subsequent adiabatic reactor, producing a reaction product;

(e) optionally repeating steps (c) and (d) in sequence one or more times; and

(f) recovering a final product, wherein the final product is the reaction product produced in a final adiabatic reactor, which is a subsequent adiabatic reactor having no subsequent adiabatic reactor in the adiabatic reaction zone downstream from the final adiabatic reactor. The final product comprises a haloolefin.

In the process disclosed herein, there is provided an adiabatic reaction zone comprising at least two serially-connected adiabatic reactors (step (a)). A starting material comprising a hydrohaloalkane is introduced to a first adiabatic reactor in the adiabatic reaction zone (step (b)).

Optionally, the process further comprises prior to step (b), a step (a′) of introducing a starting material comprising a hydrohaloalkane into a heat exchanger in the adiabatic reaction zone upstream of the first adiabatic reactor to produce a heated starting material. The heated starting material from step (a′) is the starting material introduced to the first adiabatic reactor in step (b).

Optionally the starting material may comprise other components. Alternatively, other components may be introduced to the first adiabatic reactor separately from the starting material.

Thereafter, the reaction product from the first adiabatic reactor is passed through a heat exchanger, providing an intermediate product (step (c)). The intermediate product is then introduced to a subsequent adiabatic reactor (step (d)), producing a reaction product, the process being continued to achieve a desired conversion of the hydrohaloalkane or other desired result.

Optionally the process disclosed herein comprises repeating steps (c) and (d) one or more times. In one embodiment, steps (c) and (d) are performed one to nine times, that is, steps (c) and (d) are repeated zero to eight times, so that the adiabatic reaction zone has a total of two to ten adiabatic reactors connected in series. When steps (c) and (d) are repeated one time, the reaction zone has a total of three reactors: a first adiabatic reactor, a second adiabatic reactor and a final adiabatic reactor. Accordingly, the second and final adiabatic reactors are each a subsequent reactor in step (d).

In one option of the process disclosed herein, steps (c) and (d) are not repeated and the adiabatic reaction zone consists of two adiabatic reactors—a first adiabatic reactor and a final (subsequent) adiabatic reactor.

The process further comprises recovering a final product, wherein the final product is the reaction product produced in the final adiabatic reactor.

As set forth herein, the adiabatic reactors are arranged in series with heat exchangers disposed between two serially-connected reactors in the adiabatic reaction zone. Thus, a first adiabatic reactor has no preceding reactor and the final adiabatic reactor has no subsequent reactor in the adiabatic reaction zone. Similarly, the adiabatic reaction zone contains at least a first adiabatic reactor and a final adiabatic reactor, or, in other words, at least one preceding reactor—the first adiabatic reactor—and at least one subsequent reactor—the final adiabatic reactor. A heat exchanger is upstream of and in fluid communication with each subsequent reactor.

A hydrohaloalkane may have the formula Y¹Y²CH—CXY³Y⁴, where X is halo and each Yⁱ, wherein i is 1, 2, 3 and 4, is independently H, halo, alkyl or haloalkyl, wherein halo is F, Cl, Br, or I, provided that at least one Yⁱ is halo or haloalkyl. A haloolefin may have the formula Y¹Y²C═CY³Y⁴.

### BRIEF DESCRIPTION OF THE FIGURES

FIG. 1 is a flow diagram illustrating a dehydrohalogenation process of the prior art wherein a reaction zone has a single multi-tubular reactor, which operates isothermally.

FIG. 2 is a flow diagram illustrating one embodiment of a dehydrohalogenation process of this disclosure wherein an adiabatic reaction zone and has three adiabatic reactors with a heat exchangers arranged upstream of and in fluid communication with each subsequent adiabatic reactor.

### DETAILED DESCRIPTION

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

When an amount, concentration, or other value or parameter is given as either a range, preferred range or a list of upper preferable values and/or lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range.

Before addressing details of embodiments described below, some terms are defined or clarified.

The term “haloolefin”, as used herein, means a molecule containing carbon, fluorine and/or chlorine and/or bromine and/or iodine, and a carbon-carbon double bond. Examples are described throughout the instant specification.

The term “hydrohaloolefin”, as used herein, means a molecule containing hydrogen, carbon, fluorine and/or chlorine and/or bromine and/or iodine, and a carbon-carbon double bond (halo=fluoro, chloro, bromo, iodo). Examples are described throughout the instant specification. Hydrofluoroolefin may be designated as “HFO”. Hydrochlorofluoroolefin may be designated as “HCFO”.

It should be recognized by those skilled in the art that certain haloolefins and certain hydrohaloolefins have configurational (E- and Z-) isomers. The products as produced herein thus may contain one or both of configurational isomers. The relative amounts of the configurational isomers may vary depending on reaction conditions.

The term “hydrohaloalkane”, as used herein means a molecule containing hydrogen, carbon, fluorine and/or chlorine and/or bromine and/or iodine, with no carbon-carbon double bond (halo=fluoro, chloro, bromo, iodo). Examples are described throughout the instant specification.

The term “dehydrohalogenation”, as used herein, means loss of HX from a hydrohaloalkane, where X=F, Cl, Br, I, where H and X are on adjacent carbons in the hydrohaloalkane. For example, the term “dehydrofluorination”, “dehydrofluorinating” or “dehydrofluorinated”, as used herein, means a process during which hydrogen and fluorine on adjacent carbons in a molecule are removed; the term “dehydrochlorination”, “dehydrochlorinating”, or “dehydrochlorinated”, as used herein, means a process during which hydrogen and chlorine on adjacent carbons in a molecule are removed.

The term “adiabatic”, as used herein means relating to or denoting a reactor or process or condition in a reaction zone in which heat is not intentionally added or removed from the reaction zone. It will be appreciated by those skilled in the art that even with the best insulation, some heat may be lost from reaction zones operating above ambient temperature (or conversely gained for reaction zones operating below ambient temperature).

The term “preceding adiabatic reactor” or “preceding reactor”, as used herein, means an adiabatic reactor having no adiabatic reactor upstream of this reactor in the adiabatic reaction zone. The term “subsequent adiabatic reactor” or “subsequent reactor”, as used herein, means an adiabatic reactor having at least one adiabatic reactor upstream of this reactor in the adiabatic reaction zone. The term “final adiabatic reactor” or “final adiabatic reactor”, as used herein, means an adiabatic reactor having no adiabatic reactor downstream of this reactor in the adiabatic reaction zone. Notwithstanding the foregoing, there may be one or more reactors upstream or downstream of the adiabatic reactors in the adiabatic reaction zone; there may be multiple adiabatic reactions zones, for which the definitions of preceding, subsequent and final adiabatic reactors apply only to adiabatic reactors within each adiabatic reaction zone.

Compounds referred to in this disclosure may be referred to by code, based on fluorochemical naming convention, chemical structure and/or chemical name. For convenience and reference, selected compounds with codes, structures and chemical names are provided in Table 1.

TABLE 1

| Compound | Chemical formula | Chemical name |
|---|---|---|
| 142b | CF₂ClCH₃ | 1-chloro-1,1-difluoroethane |
| 152a | CF₂HCH₃ | 1,1-difluoroethane |
| 1132a | CH₂═CF₂ | vinylidene fluoride |
| 1141 | CH₂═CHF | vinyl fluoride |
| 223db | CF₃CHClCCl₃ | 1,1,1,2-tetrachloro-3,3,3-trifluoropropane |
| 224bb | CF₃CFClCHCl₂ | 1,1,2-trichloro-2,3,3,3-tetrafluoropropane |
| 225ca | CF₃CF₂CHCl₂ | 3,3-dichloro-1,1,1,2,2-pentafluoropropane |
| 234ea | CF₃CHFCHCl₂ | 1,1-dichloro-2,3,3,3-tetrafluoropropane |
| 234bb | CF₃CFClCH₂Cl | 2,3-dichloro-1,1,1,2-tetrafluoropropane |
| 235bb | CF₃CFClCH₂F | 2-chloro-1,1,1,2,3-pentafluoropropane |
| 235cb | CF₃CF₂CH₂Cl | 3-chloro-1,1,1,2,2-pentafluoropropane |
| 235da | CF₃CHClCHF₂ | 2-chloro-1,1,1,3,3-pentafluoropropane |
| 235ea | CF₃CHFCHFCl | 3-chloro-1,1,1,2,3-pentafluoropropane |
| 235fa | CF₃CH₂CF₂Cl | 1-chloro-1,1,3,3,3-pentafluoropropane |
| 236cb | CF₃CF₂CH₂F | 1,1,1,2,2,3-hexafluoropropane |
| 236ea | CF₃CHFCHF₂ | 1,1,1,2,3,3-hexafluoropropane |
| 240aa | CH₂ClCCl₂CHCl₂ | 1,1,2,2,3-pentachloropropane |
| 240db | CCl₃CHClCH₂Cl | 1,1,1,2,3-pentachloropropane |
| 240fa | CCl₃CH₂CHCl₂ | 1,1,1,3,3-pentachloropropane |
| 243ab | CF₃CCl₂CH₃ | 2,2-dichloro-1,1,1-trifluoropropane |
| 243db | CF₃CHClCH₂Cl | 2,3-dichloro-1,1,1-trifluoropropane |
| 243fa | CF₃CH₂CHCl₂ | 1,1-dichloro-3,3,3-trifluoropropane |
| 243fb | CF₂ClCH₂CHFCl | 1,3-dichloro-1,1,3-trifluoropropane |
| 244bb | CF₃CFClCH₃ | 2-chloro-1,1,1,2-tetrafluoropropane |
| 244db | CF₃CHClCH₂F | 2-chloro-1,1,1,3-tetrafluoropropane |
| 244eb | CF₃CHFCH₂Cl | 3-chloro-1,1,1,2-tetrafluoropropane |
| 244fa | CF₃CH₂CHFCl | 3-chloro-1,1,1,3-tetrafluoropropane |
| 245cb | CF₃CF₂CH₃ | 1,1,1,2,2-pentafluoropropane |
| 245eb | CF₃CHFCH₂F | 1,1,1,2,3-pentafluoropropane |
| 245fa | CF₃CH₂CHF₂ | 1,1,1,3,3-pentafluoropropane |
| 250fb | CCl₃CH₂CH₂Cl | 1,1,1,3-tetrachloropropane |
| 253db | CF₃CHClCH₃ | 2-chloro-1,1,1-trifluoropropane |
| 253fb | CF₃CH₂CH₂Cl | 1-chloro-3,3,3-trifluoropropane |
| 254fb | CF₃CH₂CH₂F | 1,1,1,3-tetrafluoropropane |
| 336mdd | CF₃CHClCHClCF₃ | 2,3-dichloro-1,1,1,4,4,4-hexafluorobutane |
| 336mfa | CF₃CCl₂CH₂CF₃ | 2,2-dichloro-1,1,1,4,4,4-hexafluorobutane |
| 346mdf | CF₃CHClCH₂CF₃ | 2-chloro-1,1,1,4,4,4-hexafluorobutane |
| 1213xa | CF₃CCl═CCl₂ | 1,1,2-trichloro-3,3,3-trifluoropropene |
| 1214ya | CF₃CF═CCl₂ | 1,1-dichloro-2,3,3,3-tetrafluoropropene |
| 1224yd | E- and/or Z- | 1-chloro-2,3,3,3-tetrafluoropropene |
| | CF₃CF═CHCl | |
| 1225yc | CHF₂CF═CF₂ | 1,1,2,3,3-pentafluoropropene |
| 1225ye | E- and/or Z- | E- or Z-1,2,3,3,3-pentafluoropropene |
| | CF₃CF═CHF | |
| 1225zc | CF₃CH═CF₂ | 1,1,3,3,3-pentafluoropropene |
| 1230xa | CCl₂═CClCH₂Cl | 1,1,2,3-tetrachloropropene |
| 1233xf | CF₃CCl═CH₂ | 2-chloro-3,3,3-trifluoropropene |
| 1233zd | E- and/or Z- | 1-chloro-3,3,3-trifluoropropene |
| | CF₃CH═CHCl | |
| 1234yf | CF₃CF═CH₂ | 2,3,3,3-tetrafluoropropene |
| 1234ze | E- and/or Z- | E- or Z-1,3,3,3-tetrafluoropropene |
| | CF₃CH═CHF | |
| 1243zf | CF₃CH═CH₂ | 3,3,3-trifluoropropene |
| 1326mxz | E- and/or Z- | 1,1,1,4,4,4-hexafluoro-2-chloro-2-butene |
| | CF₃CCl═CHCF₃ | |
| 1336mzz | E- and/or Z- | 1,1,1,4,4,4-hexafluoro-2-butene |
| | CF₃CH═CHCF₃ | |

The present disclosure provides a process for dehydrohalogenating a hydrohaloalkane, which process comprises the steps: (a) providing an adiabatic reaction zone comprising at least two serially-connected adiabatic reactors and having a heat exchanger disposed in sequence and in fluid communication between each two reactors in series; (b) introducing a starting material comprising a hydrohaloalkane into a first adiabatic reactor of the serially-connected reactors, producing a reaction product; (c) passing the reaction product from a preceding adiabatic reactor to a heat exchanger, producing an intermediate product; (d) introducing the intermediate product from the heat exchanger to a subsequent adiabatic reactor, producing a reaction product; optionally repeating steps (c) and (d) one or more times; and (e) recovering a final product, wherein the final product is the reaction product produced in a final adiabatic reactor, which is a subsequent adiabatic reactor having no subsequent adiabatic reactor in the adiabatic reaction zone downstream from the final adiabatic reactor. In step (c), the heat exchanger is downstream from and in fluid communication with the preceding adiabatic reactor.

The present disclosure provides a process for dehydrohalogenating a starting material comprising a hydrohaloalkane to produce a final product comprising a haloolefin.

A hydrohaloalkane has the formula Y¹Y²CH—CXY³Y⁴, where X is F, Cl, Br or I and each of Yⁱ, wherein i is 1, 2, 3 and 4, is independently chosen from H, F, Cl, Br, I, an alkyl group or a haloalkyl group, provided that at least one Yⁱ is not H or at least one Yⁱ is a haloalkyl group, wherein a haloalkyl is a fluoroalkyl, a chloroalkyl, a bromoalkyl or an iodoalkyl, that is, halo=fluoro, chloro, bromo, or iodo. In some embodiments the alkyl group is a C₁ to C₃ alkyl group. In some embodiments the haloalkyl group is a C₁ to C₃ haloalkyl group. The corresponding haloolefin has the formula, Y¹Y²C═CY³Y⁴.

A hydrohaloalkane may be or comprise a hydrochloroalkane (containing H, Cl and C). A hydrohaloalkane may be or comprise a hydrofluorochloroalkane (containing H, F, Cl and C). A hydrohaloalkane may be or comprise a hydrofluoroalkane (containing H, F and C). Bromo- and iodo-containing hydrohaloalkanes are also contemplated herein.

In some embodiments, the present disclosure provides a process for making at least one haloethene (haloethylene) product from a starting material comprising hydrohaloethane. A hydrohaloethane may have the formula Y¹Y²CH—CXY³Y⁴, where X is halo and each Yⁱ (i is 1, 2, 3 and 4) is independently H or halo, halo being F, Cl, Br, I, provided that at least one Yⁱ is halo. Example of hydrohaloethane is 1-chloro-1,1-difluoroethane (CF₂ClCH₃) and example of haloethylene is vinylidene fluoride (CF₂═CH₂). A second example of hydrohaloethane is 1,1-difluoroethane (CHF₂CH₃) and example of haloethylene is vinyl fluoride (CHF═CH₂).

The present disclosure provides a process for making at least one halopropene product from a starting material comprising a hydrohalopropane. A hydrohalopropane has the formula Y¹Y²CH—CXY³Y⁴, where X is halo and three Yⁱ (i is 1, 2, 3 and 4) are independently H or halo and the other Yⁱ is C₁ alkyl or C₁ haloalkyl, where halo is F, Cl, Br, or I, provided further that that at least one Yⁱ is halo or haloalkyl.

Representative hydrohalopropanes include CF₃CFClCH₃, CF₃CHFCH₂Cl, CF₃CHClCH₂F, CF₃CH₂CHFCl, CF₃CHFCH₂F, CF₃CH₂CHF₂, CF₃CF₂CH₃, CF₃CFClCH₂F, CF₃CHFCHFCl, CF₃CHClCHF₂, CF₃CH₂CF₂Cl, CF₃CHClCH₃, CF₃CHClCH₂Cl, CF₃CH₂CH₂Cl, CCl₃CH₂CHCl₂, CCl₃CHClCH₂Cl, CCl₃CH₂CH₂Cl, CH₂ClCCl₂CHCl₂, and mixtures of two or more thereof.

The hydrohalopropane may be or comprise a hydrochloropropane. The hydrochloropropane may be or comprise CCl₃CHClCH₂Cl, CCl₃CH₂CHCl₂, CCl₃CH₂CH₂Cl, or mixtures of two or more thereof.

The hydrohalopropane may be or comprise a hydrochlorofluoropropane. The hydrochlorofluoropropane may be or comprise CF₃CHClCCl₃, CF₃CFClCHCl₂, CF₃CF₂CHCl₂, CF₃CHFCHCl₂, CF₃CFClCH₂Cl, CF₃CF₂CH₂Cl, CF₃CHFCHFCl, CF₃CHClCHF₂, CF₃CH₂CF₂Cl, CF₃CCl₂CH₃, CF₃CHClCH₂Cl, CF₃CH₂CHCl₂, CF₂ClCH₂CHFCl, CF₃CFClCH₃, CF₃CHClCH₂F, CF₃CHFCH₂Cl, CF₃CH₂CHFCl, CF₃CHClCH₃, CF₃CH₂CH₂Cl, or mixtures of two or more thereof. In one embodiment, the hydrochlorofluoropropane is CF₃CFClCH₃.

The hydrohalopropane may be or comprise a hydrofluoropropane. The hydrofluoropropane may be or comprise CF₃CF₂CH₂F, CF₃CHFCHF₂, CF₃CF₂CH₃, CF₃CHFCH₂F, CF₃CH₂CHF₂, CF₃CH₂CH₂F, or mixtures of two or more thereof. The hydrohalopropane may be a hydrofluoropropane. The hydrofluoropropane may be CF₃CHFCH₂F, or CF₃CH₂CHF₂, or CF₃CF₂CH₃, or mixtures of two or more thereof.

In one embodiment, the starting material comprises a hydrohalopropane having the formula CF₃CFQCH₃, where Q is Cl or F. The starting material may comprise CF₃CF₂CH₃. The starting material may comprise CF₃CFClCH₃. Dehydrohalogenation of CF₃CFClCH₃ produces a product comprising CF₃CF═CH₂. Dehydrohalogenation of CF₃CFClCH₃ may produce a product comprising a mixture of CF₃CF═CH₂ and E-CF₃CH═CHF and Z—CF₃CH═CHF.

In one embodiment, dehydrohalogenation of a hydrohalopropane produces a product comprising a halopropene. In particular embodiment, the product comprises a chloropropene. In another embodiment, the product comprises a fluorochloropropene. In another embodiment, the product comprises a fluoropropene.

In an embodiment, a hydrohalopropane is or comprises CH₂ClCHClCCl₃ and a chloropropene is or comprises CH₂ClCCl═Cl₂ (240db→1230xa).

In an embodiment, a hydrohalopropane is or comprises CF₃CFClCH₃ and a hydrofluoropropene is or comprises CF₃CF═CH₂ (244bb→1234yf).

In another embodiment, a hydrohalopropane is or comprises CF₃CHFCH₂Cl and halopropene is or comprises CF₃CF═CH₂ (244eb→1234yf).

In another embodiment, a hydrohalopropane is or comprises CF₃CHClCH₂F and a halopropene is or comprises E- and/or Z—CF₃CH═CHF (244db→1234ze).

In another embodiment, a hydrohalopropane is or comprises CF₃CH₂CHFCl and a halopropene is or comprises E- and/or Z—CF₃CH═CHF (244fa→1234ze).

In another embodiment, a hydrohalopropane is or comprises CF₃CFClCH₂F and a halopropene is or comprises E- and/or Z—CF₃CF═CHF (235bb→1225ye).

In another embodiment, a hydrohalopropane is or comprises CF₃CF₂CH₂Cl and a halopropene is or comprises E- and/or Z—CF₃CF═CHCl (235cb→1224yd).

In another embodiment, a hydrohalopropane is or comprises CF₃CHClCHF₂ and a halopropene is or comprises CF₃CH═CF₂ (235da→1225zc).

In another embodiment, a hydrohalopropane is or comprises CF₃CH₂CF₂Cl and a halopropene is or comprises CF₃CH═CF₂ (235fa→1225zc).

In another embodiment, a hydrohalopropane is or comprises CF₃CHClCCl₃ and a halopropene is or comprises CF₃CCl═CCl₂ (223db→1213xa).

In another embodiment, a hydrohalopropane is or comprises CF₃CHClCH₂Cl and a halopropene is or comprises CF₃CCl═CH₂ (243db→1233xf).

In another embodiment, a hydrohalopropane is or comprises CF₃CH₂CHCl₂ and a halopropene is or comprises E- and/or Z—CF₃CH═CHCl (243fa→1233zd).

In another embodiment, the hydrohalopropane is or comprises CF₃CH₂CH₂Cl and a halopropene is or comprises CF₃CH═CH₂ (253fb→1243zf).

In a particular embodiment a hydrohalopropane is or comprises CF₃CF₂CH₂F and a halopropene is or comprises E- and/or Z—CF₃CF═CHF (236cb→1225ye).

In another embodiment a hydrohalopropane is or comprises CF₃CHFCHF₂ and a halopropene is or comprises E- and/or Z—CF₃CF═CHF (236ea→1225ye).

In another embodiment a hydrohalopropane is or comprises CF₃CF₂CH₃ and a halopropene is or comprises CF₃CH═CH₂ (245cb→1234yf).

In another embodiment a hydrohalopropane is or comprises CF₃CHFCH₂F and a halopropene is or comprises CF₃CH═CH₂ (245eb→1234yf).

In another embodiment the hydrohalopropane is or comprises CF₃CH₂CHF₂ and the halopropene is or comprises E- and/or Z—CF₃CH═CHF (245fa→1234ze).

In one embodiment, the hydrohaloalkane is or comprises a hydrochloropropane, which undergoes hydrofluorination and dehydrohalogenation in the presence of HF and a catalyst, forming a fluoro(chloro)propene. In a particular embodiment, a hydrochloropropane is or comprises 1,1,1,3-tetrachloropropane (250fb), and the product from dehydrohalogenation comprises 3,3,3-trifluoropropene (1243zf).

When the haloolefin is 1243zf, the process optionally further comprises chlorinating 1243zf to produce a product comprising 243db, dehydrochlorinating 243db to produce a product comprising 1233xf, hydrofluorinating 1233xf to produce a product comprising 244bb, and dehydrochlorinating 244bb to produce a product comprising 1234yf. Optionally, the process further comprises purifying each product. Thus, in this example, the process may further comprise purifying a product comprising 1243zf, a product comprising 243db, a product comprising 1233xf, a product comprising 244bb, a product comprising 1234yf, or two or more of the products.

When the haloolefin is 1225ye, the process optionally further comprises hydrogenating 1225ye to produce a product comprising 245eb, and dehydrofluorinating 245eb to produce a product comprising 1234yf. Optionally, the process further comprises purifying the product comprising 1225ye and/or the product comprising 245eb.

When the haloolefin is 1225zc, the process optionally further comprises hydrogenating 1225zc to produce a product comprising 245fa, and dehydrofluorinating 245fa to produce a product comprising E- and/or Z-1234ze. Optionally, the process further comprises purifying the product comprising 245fa and/or the product comprising E- and/or Z-1234ze.

When the haloolefin is 1233xf, the process optionally further comprises hydrofluorinating 1233xf to produce a product comprising 244bb, and dehydrochlorinating 244bb to produce a product comprising 1234yf. Optionally, the process further comprises purifying the product comprising 1233xf and/or purifying the product comprising 244bb and/or purifying the product comprising 1234yf.

When a product comprising 1234yf is produced, the process optionally further comprises purifying the product comprising 1234yf.

The present disclosure provides a process for making at least one hydrohalobutene product from a starting material comprising hydrohalobutane. A hydrohalobutane may have the formula Y¹Y²CH—CXY³Y⁴, where X is halo and two Yⁱ (i is 1, 2, 3 and 4) are C₁ alkyl or C₁ haloalkyl, and the remaining two Yⁱ are independently H or halo; or one Yⁱ is a C₂ alkyl or a C₂ haloalkyl and the remaining three Yⁱ are each independently H or halo, where halo is F, Cl, Br, or I, provided that at least one Yⁱ is halo or a haloalkyl.

Representative hydrohalobutanes include CF₃CHClCHClCF₃, CF₃CCl₂CH₂CF₃, CF₃CH₂CHClCF₃ and mixtures thereof. Examples of halobutenes include CF₃CCl═CHCF₃ and E- and/or Z—CF₃CH═CHCF₃.

Dehydrohalogenation of a hydrohalobutane produces a product comprising a halobutene. In one embodiment, a hydrohalobutane is or comprises a hydrochlorofluorobutane and a halobutene is or comprises a fluorobutene.

In a particular embodiment a halobutane is or comprises CF₃CHClCHClCF₃ and a halobutene is or comprises E- and/or Z—CF₃CCl═CHCF₃ (336mdd→1326mxz).

In another embodiment a halobutane is or comprises CF₃CCl₂CH₂CF₃ and a halobutene is or comprises E- and/or Z—CF₃CCl═CHCF₃. (336mfa→1326mxz).

In one embodiment, a halobutane is or comprises CF₃CHClCH₂CF₃ and a halobutene is or comprises E- and/or Z—CF₃CH═CHCF₃ (346mdf→1336mzz).

The present disclosure provides a process for making at least one hydrohalopentene product from a starting material comprising hydrohalopentane. A hydrohalopentane may have the formula Y¹Y²CH—CXY³Y⁴, where X is halo and three Yⁱ are C₁ alkyl or C₁ haloalkyl group and the other Yⁱ is H or halo; or one Yⁱ is C₂ alkyl or C₂ haloalkyl group and one Yⁱ is C₁ alkyl or C₁ haloalkyl group and the other Yⁱ is H or halo; or one Yⁱ (i is 1, 2, 3 and 4) is C₃ alkyl or C₃ haloalkyl group and the other Yⁱ is H or halo; and where halo is F, Cl, Br, or I, provided that at least one Yⁱ is halo or a haloalkyl.

Hydrohalopentane may be chosen from CF₃CCl₂CH₂C₂F₅, CF₃CHClCHClC₂F₅, CF₃CHClCH₂C₂F₅, CF₃CF(CF₃)CFClCH₃, and mixtures thereof. Examples of halopentenes include CF₃CCl═CHC₂F₅, CF₃CH═CHC₂F₅, and CF₃CF(CF₃)CF═CH₂.

Higher haloalkenes may also be produced using the processes disclosed herein.

A dehydrohalogenating step is carried out in an adiabatic reaction zone. The adiabatic reaction zone comprises at least two serially-connected adiabatic reactors and having a heat exchanger in fluid communication disposed between each two reactors in series.

The adiabatic reaction zone comprises a first adiabatic reactor and a final adiabatic reactor. The first adiabatic reactor is a preceding adiabatic reactor relative to any adiabatic reactors or heat exchangers downstream from the first adiabatic reactor in the adiabatic reaction zone. The final adiabatic reactor is a subsequent adiabatic reactor relative to any adiabatic reactors or heat exchangers upstream of the final adiabatic reactor in the adiabatic reaction zone.

The first adiabatic reactor is upstream of and in fluid communication with a heat exchanger. The heat exchanger is in fluid communication and upstream of a subsequent adiabatic reactor.

In one embodiment, the adiabatic reaction zone consists of two reactors, a first adiabatic reactor and a final adiabatic reactor. In this embodiment, a heat exchanger is downstream from the first adiabatic reactor and upstream of the final adiabatic reactor.

One skilled in the art will understand the relationships between the first adiabatic reactor, which has no preceding (upstream) reactor, a subsequent adiabatic reactor, which has at least one preceding reactor and the final adiabatic reactor, which has no subsequent (downstream) reactor and is a subsequent reactor in step (c). The adiabatic reactors in the adiabatic reaction zone are in fluid communication with heat exchangers, wherein a heat exchanger is disposed between two reactors.

In an embodiment, the adiabatic reaction zone consists of a first adiabatic reactor, a second adiabatic reactor (which may also be referred to as a subsequent adiabatic reactor) and a final adiabatic reactor, which is also a subsequent adiabatic reactor in accordance with step (d) of the process disclosed herein, thus, a total of three reactors, where each reactor operates adiabatically and a heat exchanger is arranged between the first adiabatic reactor and the second adiabatic reactor and a heat exchanger is arranged between the second adiabatic reactor and the final adiabatic reactor. Thus, steps (c) and (d) are repeated once. A person skilled in the art is able to contemplate using more than three reactors, such as repeating steps (c) and (d) two times or three times or more.

An upper limit of the number of adiabatic reactors and heat exchangers, where a heat exchanger is disposed between two reactors in the adiabatic reaction zone may be based on practical reasons such as controlling cost and complexity or based on achieving a particular goal such as conversion of starting material or a formation of a particular product. Two or more adiabatic reactors are used in the adiabatic reaction zone, for example two to ten reactors (repeat steps (c) and (d) zero to eight times), or two to four reactors (repeat steps (c) and (d) zero to two times).

The adiabatic reactors may be of any shape that is conducive to performing the dehydrohalogenation process as disclosed herein. In certain embodiments, each reactor is a cylindrical tube or pipe, which may be straight or coiled. A plug flow design is preferable because it minimizes back mixing which results in lower overall conversion.

Due to the corrosive nature of the dehydrohalogenation process as set forth herein, adiabatic reactors for use in the adiabatic reaction zone disclosed herein are comprised of materials which are resistant to corrosion. Such materials include stainless steel, in particular of the austenitic type or copper-clad steel or nickel-based alloy or gold or gold-lined or quartz. Nickel-based alloys are available commercially and include, for example, high nickel alloys, such as Monel™ nickel-copper alloys, Hastelloy™ nickel-based alloys and, Inconel™ nickel-chromium alloys. In one embodiment, the reactor is comprised of nickel-based alloy. Adiabatic reactors may be lined with fluoropolymer, provided the fluoropolymer is compatible with the temperature. Other materials may include SiC or graphite for corrosion resistance.

In addition to the adiabatic reactors of the adiabatic reaction zone disclosed herein, heat exchangers, effluent lines, units associated with mass transfer, contacting vessels (pre-mixers), distillation columns, and feed and material transfer lines associated with reactors, heat exchangers, vessels, columns, and units that are used in the processes of embodiments disclosed herein should be constructed of materials resistant to corrosion, such as those recited above.

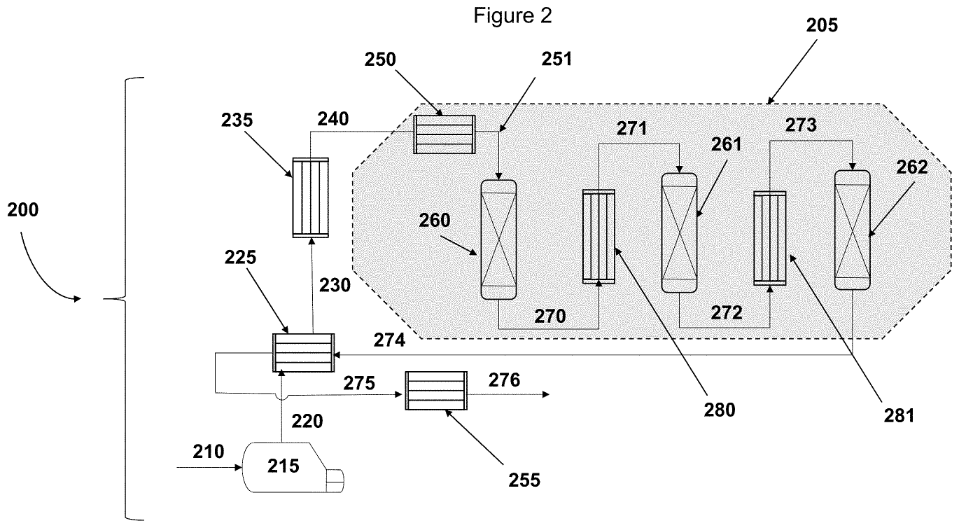

The present disclosure provides an adiabatic reaction zone. The adiabatic reaction zone comprises at least two adiabatic reactors. A heat exchanger is arranged between each two reactors (see also below, discussion of FIG. 2). In one embodiment of the process disclosed herein, the process comprises providing an adiabatic reaction zone comprising at least two serially-connected adiabatic reactors and having a heat exchanger disposed in sequence and in fluid communication between each two reactors in series; introducing a starting material comprising a hydrohaloalkane into an adiabatic reaction zone wherein a first reaction product is produced in a first adiabatic reactor; passing the first reaction product from the first adiabatic reactor to a heat exchanger to produce an intermediate product; then introducing the intermediate product from the heat exchanger to a subsequent adiabatic reactor wherein a second reaction product is produced; and optionally, passing the second reaction product from the subsequent adiabatic reactor through a heat exchanger prior to introducing the second reaction product into a third adiabatic reactor, if present, and so on.

Notwithstanding the foregoing, other process steps may occur upstream of the adiabatic reaction zone. The upstream process steps may involve, for example, a process to prepare the hydrohaloalkane for use in the dehydrohalogenation process as set forth herein or vaporization of a starting material to be fed to the first adiabatic reactor. The upstream process steps may be performed in one or more reactors. For clarity, even if one or more reactors are present upstream of the dehydrohalogenation process, the “first adiabatic reactor” referred to herein refers to a first adiabatic reactor in a series of adiabatic reactors in which the dehydrohalogenation process is performed wherein a heat exchanger is located between the first adiabatic reactor in the series and the second (subsequent) reactor in the series. Thus, any reactors in which process steps are performed upstream of the adiabatic reaction zone and thus upstream of the first adiabatic reactor as thus defined, cannot be considered the “first adiabatic reactor”.

There may be other reactions (processes and reaction zones) which occur downstream from the dehydrohalogenation process in the adiabatic reaction zone as set forth herein.

Heat exchangers are used in the process and adiabatic reaction zone of the present disclosure. A heat exchanger is arranged between two adiabatic reactors in series. Heat exchangers replace the heat used by the reaction as dehydrohalogenation is an endothermic process. Heat exchangers used herein may be shell and tube heat exchangers. Heat exchangers may employ fin and tube heat exchangers, microchannel heat exchangers and vertical or horizontal single pass tube or plate type heat exchangers, electric heaters, among others. Heat exchangers may provide heat by electric heating. Heat exchangers may use process streams as heat exchange fluid. Other designs may be used which are compatible with the physical and chemical requirements of the process, including the temperature and corrosive nature of the reaction components.

Each heat exchanger may represent multiple heat exchangers in sequence, where multiple means more than one heat exchanger. Multiple heat exchangers may be used in the event that multiple heat sources are available, but certain heat sources (such as steam) may not be capable of heating to desired temperatures for pyrolytic or adiabatic reactions.

In one embodiment, each heat exchanger may be operated independently of the other heat exchangers in the adiabatic reaction zone. Each heat exchanger may be operated to provide an intermediate product having the same temperature as the intermediate product exiting another heat exchanger in the adiabatic reaction zone.

In another embodiment, each heat exchanger may be operated to provide an intermediate product having a different temperature relative to intermediate products exiting other heat exchangers in the adiabatic reaction zone.

In one embodiment, each adiabatic reactor in the reaction zone is operated at the same temperature. In another embodiment, at least one adiabatic reactor in the adiabatic reaction zone is operated at a different temperature than the other adiabatic reactor(s) in the adiabatic reaction zone. It should be understood that if the adiabatic reaction zone consists of two adiabatic reactors, each reactor may operate at the same temperature or at different temperatures and if the adiabatic reaction zone consists of more than two adiabatic reactors, each reactor may operate independently at the same or at a different temperature from each other reactor in the adiabatic reaction zone.

In one embodiment, the adiabatic reactors in the adiabatic reaction zone operate at different temperatures. For example a first adiabatic reactor may operate at a higher temperature than a subsequent adiabatic reactor. It has been surprisingly found that by operating a first adiabatic reactor at a different temperature than a subsequent adiabatic reactor the product profile varies. Thus, if certain secondary products are more desired than other secondary products for any reason (such as, for example, ease of separation from main product, commercial value of secondary products, among other reasons), the adiabatic reactors may be operated at different temperatures. In one embodiment, the first or a preceding adiabatic reactor operates at a temperature higher than a subsequent adiabatic reactor, contemplating two or more adiabatic reactors in the adiabatic reaction zone.

In one embodiment, each heat exchanger may be independently disposed in a vessel with the preceding or subsequent adiabatic reactor. In another embodiment, each heat exchanger may be independently disposed in a separate vessel from the preceding or subsequent adiabatic reactor. Fluid communication is maintained between subsequent adiabatic reactors through heat exchangers as set forth previously.

Optionally, a heat exchanger may also be used to heat starting material to desired reaction temperature upstream of the first adiabatic reactor either in the adiabatic reaction zone or external to the adiabatic reaction zone. In one embodiment a heat exchanger is upstream of the first adiabatic reactor in the adiabatic reaction zone. In such embodiment, the process comprises a step (a′) of introducing a starting material comprising a hydrohaloalkane into a heat exchanger in the adiabatic reaction zone upstream of the first adiabatic reactor to produce a heated starting material. The heated starting material from step (a′) is the starting material introduced to the first adiabatic reactor in step (b).

The processes and adiabatic reaction zone disclosed herein provide greater reactor volume to accommodate for relatively slow dehydrohalogenation reactions. The total reactor volume increases relative to multi-tubular reactors without the complexity while controlling temperature for endothermic processes.

The process disclosed herein is performed in the gas phase in the presence (catalytic process) or absence of an added catalyst (pyrolysis process) at a temperature sufficient to effect conversion of the hydrohaloalkane to a haloolefin (haloalkene) in the reaction zone.

Each of the adiabatic reactors of the adiabatic reaction zone disclosed herein may independently operate as a catalytic or pyrolytic adiabatic reactor. That is, each reactor may be catalytic or each reactor may be pyrolytic or a combination of catalytic and pyrolytic reactors may be used. More specificity is provided below with respect to options for a pyrolysis process and suitable catalysts for a catalytic process.

In some embodiments, whether a catalytic process or a pyrolysis process, an inert diluent gas (optional component) is used as a carrier gas for the hydro(chloro)fluoropropane. In one embodiment, the carrier gas is selected from nitrogen, argon, helium or carbon dioxide. In addition a carrier gas may include unconverted starting material in a reactor other than the first adiabatic reactor, recycled product, HF, HCl, among others. The carrier gas may include an organic material that does not negatively impact the process chemistry.

In one embodiment, at least one adiabatic reactor operates as a pyrolysis reactor. That is, the adiabatic reaction zone comprises one or more adiabatic reactors which operate as a pyrolysis reactor. In such an embodiment, the process is performed by pyrolyzing (thermally dehydrohalogenating) the starting material to produce the hydrofluoroolefin product, that is, pyrolysis. The term “pyrolyzing” or “pyrolysis”, as used herein, means chemical change produced by heating in the absence of added catalyst. By “absence of added catalyst” is meant that no material is added to the adiabatic reactor to purposefully increase the reaction rate by reducing the activation energy of the dehydrohalogenation process. Notwithstanding the foregoing, the surface of an adiabatic reactor may have some catalytic properties.

When the dehydrohalogenation process is a pyrolysis process, the flow of gases through the pyrolysis reactor may be passed through perforated baffles into the reactor, such as, for example to create a uniform flow distribution that approaches plug flow. Plug flow is desired as backmixing reduces conversion.

In other embodiments, an adiabatic pyrolysis reactor is substantially empty, which means that the free volume of the adiabatic reaction zone is at least about 80%, and in another embodiment, at least about 90%, and in another embodiment at least about 95%. The free volume is the volume of the reaction zone minus the volume of the material that makes up the reactor packing, and free volume may be expressed as a percent (%) as the ratio of the free volume relative to the total volume of the reactor times 100.

The dehydrohalogenation process of this disclosure may include a dehydrofluorination process or a dehydrochlorination process or both a dehydrofluorination and a dehydrochlorination process depending on the starting material and the corresponding fluoroolefin product. For example, when the hydrohaloalkane is 244bb, dehydrochlorination produces 1234yf. However, reaction conditions may also result in some dehydrofluorination to 1233xf.

Typically, the pyrolysis temperature for dehydrofluorination is higher than the pyrolysis temperature for dehydrochlorination. In certain embodiments, the process is a dehydrofluorination process and a pyrolysis reactor is operated at a temperature of from about 500° C. to about 900° C. In certain embodiments, the process is a dehydrochlorination process and an adiabatic pyrolysis reactor is operated at a temperature of from about 300° C. to about 700° C. Pyrolysis processes have also been disclosed, for example, in U.S. Pat. Nos. 7,833,434; 8,203,022; and 8,445,735.

The dehydrohalogenation process of this disclosure may have a reaction pressure that is subatmospheric, atmospheric or superatmospheric. In one embodiment, the process is conducted at a pressure of from about 0 psig to about 200 psig. In one embodiment, the reaction is conducted at a pressure of from 10 psig to about 150 psig. In another embodiment, the reaction is conducted at a pressure of from 20 psig to about 100 psig.

In one embodiment, each adiabatic reactor is operated as an adiabatic pyrolytic reactor.

In one embodiment, at least one adiabatic reactor in the adiabatic reaction zone operates as an adiabatic catalytic reactor. That is, the adiabatic reaction zone comprises one or more adiabatic reactors which operate as an adiabatic catalytic reactor. In such an embodiment, this catalytic adiabatic reactor is charged with a catalyst to produce the hydrofluoroolefin product. Any dehydrohalogenation catalyst may be used.

For example, the dehydrohalogenation catalyst may be chosen from metal halides, metal oxides, halogenated metal oxides, neutral (or zero oxidation state) metal or metal alloy, or carbon in bulk or supported form.

The dehydrohalogenation catalyst may be chosen from metal halide or metal oxide or metal oxyhalide catalysts including but are not limited to, mono-, bi-, and tri-valent metal halides, metal oxides, metal oxyhalides and combinations of two or more thereof, and more preferably mono-, bi-, and tri-valent metal halides and combinations of two or more thereof.

Metals include transition metals, alkali metals, alkaline earth metals. A metal halide or metal oxide or metal oxyhalide may be supported or unsupported. A metal halide or metal oxide or metal oxyhalide may be supported on carbon, alkaline earth metal halides or on alkaline earth metal oxides.

Examples of suitable metals for use in dehydrohalogenation catalysts herein include, but are not limited to, Cr³⁺, Fe³⁺, Ca²⁺, Mg²⁺, Ca²⁺, Ni²⁺, Zn²⁺, Pd²⁺, Li⁺, Na⁺, K⁺, and Cs⁺. Component halides include, but are not limited to, F⁻, Cl⁻, Br⁻, and I⁻. Examples of useful mono- or bi- or tri-valent metal halide include, but are not limited to, LiF, NaF, KF, CsF, MgF₂, CaF₂, LiCl, NaCl, KCl, CsCl, CrCl₃ and FeCl₃. Supported metal halide catalysts include fluorinated CsCl/MgO, CsCl/MgF₂ and the like.

The dehydrohalogenation catalyst may be chosen from neutral (that is, zero valent) metals, metal alloys of mixtures thereof. Useful metals include, but are not limited to, Pd, Pt, Rh, Fe, Co, Ni, Cu, Mo, Cr, Mn, and combinations of the foregoing as alloys or mixtures. A neutral metal catalyst may be supported or unsupported. Useful examples of metal alloys include, but are not limited to, stainless steel (e.g., SS 316), austenitic nickel-based alloys (e.g., Inconel 625, Inconel 660, Inconel 825, Monel 400), and the like.

Other suitable dehydrohalogenation catalysts for the dehydrohalogenation processes disclosed herein include carbon catalysts, which may be chosen from acid-washed carbon, activated carbon and three-dimensional matrix carbonaceous materials.

The dehydrohalogenation catalyst may alternatively be chosen from alumina, fluorided alumina, aluminum fluoride, aluminum chlorofluoride; metal compounds supported on alumina, fluorided alumina, aluminum fluoride, or aluminum chlorofluoride; chromium oxide (Cr₂O₃), fluorided chromium oxide, and cubic chromium trifluoride; oxides, fluorides, and oxyfluorides of magnesium, zinc and mixtures of magnesium and zinc and/or aluminum; lanthanum oxide, fluorided lanthanum oxide or mixtures thereof.

Fluorided or fluorine-containing catalysts may be charged to the catalytic reactor or precursors of fluorided or fluorine-containing catalysts may be formed in situ in the catalytic reactor by introducing HF to the reactor.

The recitation of suitable dehydrohalogenation catalysts hereinabove is meant for illustrative purposes and not intended to be comprehensive. Persons skilled in the art will appreciate other dehydrohalogenation catalysts not specifically recited herein may be used.

In a catalytic dehydrohalogenation process, the adiabatic catalytic reactor may be suitably operated at a temperature from about 150 to about 550° C. and a pressure of from about 0 to about 200 psig or from 10 to 150 psig or from 20 to 100 psig.

In one embodiment, each adiabatic reactor is operated as an adiabatic catalytic reactor.

The dehydrohalogenation process disclosed herein produces a product comprising a haloolefin. Byproduct HF or HCl may be removed by a number of methods such as distillation or washing with water to produce an aqueous HF or HCl solution or condensing and decanting an acid-rich phase or scrubbing with base to produce an acid-free organic product which, optionally, may undergo further purification using one or any combination of purification techniques that are known in the art.

In accordance with this disclosure, the process may comprise purifying the starting material, a hydrohaloalkane. When the haloolefin produced according to a process of this disclosure is an intermediate for subsequent reaction(s), the process may further comprise purifying the intermediate haloolefin product prior to subsequent reaction(s).

The process disclosed herein optionally further comprises recovering the haloolefin from the final product. The haloolefin may be recovered using processes known to those skilled in the art and with examples described in this disclosure. The process disclosed herein optionally further comprises purifying the haloolefin. Processes for recovering and/or purifying the haloolefin may include distillation, condensation, decantation, absorption into water, scrubbing with base, and combinations of two or more thereof.

In certain embodiments, various azeotropic or azeotrope-like (i.e., near azeotrope) compositions comprising the hydrofluoropropene product may be utilized in the processes of recovering and/or purifying the haloolefin or intermediate products.

In one embodiment, HF may be added to a product comprising 1234yf. In one embodiment, HF may be present in the product comprising 1234yf. In either embodiment, 1234yf and HF are combined to form an azeotrope or near azeotrope of 1234yf and HF. An azeotrope or near-azeotropic mixture of HF and 1234yf may also be formed as the distillate from a distillation column where a non-azeotropic mixture of HF and 1234yf are present in the feed. Separation of 1234yf includes isolation of the azeotrope or near azeotrope of 1234yf and HF and subjecting the azeotrope or near azeotrope of 1234yf and HF to further processing to produce HF-free 1234yf by using procedures similar to those disclosed in U.S. Pat. No. 7,897,823. Azeotrope or near azeotrope compositions of HFO-1234yf and HF have been disclosed in U.S. Pat. No. 7,476,771, and the process described therein may also be utilized for recovering the hydrofluoroolefin product.

In another embodiment, HF may be added to a product comprising E- and/or Z-1234ze, producing an azeotropic or near azeotropic composition comprising E- and/or Z-1234ze and HF. The azeotropic or near azeotropic composition comprising E- and/or Z-1234ze and HF may be isolated, e.g., by distillation for separation from other products.

The azeotropic or near azeotropic composition of E- and/or Z-1234ze and HF is subjected to further processing to produce HF-free E- and/or Z-1234ze by using procedures similar to those disclosed in U.S. Pat. No. 7,897,823.

In addition, techniques applied in U.S. Pat. Nos. 7,423,188 and 8,377,327 may be utilized to recover HF-free E- and Z-1234ze, produced according to the process disclosed herein. U.S. Pat. No. 7,423,188 discloses azeotrope or near-azeotrope compositions of the E-isomer of 1234ze and HF. U.S. Pat. No. 8,377,327 discloses azeotrope or near-azeotrope compositions of the Z-isomer of 1234ze and HF.

The present disclosure also provides a process for the preparation of 1234yf which comprises the following steps: (v) providing an adiabatic reaction zone comprising at least two serially-connected adiabatic reactors and having a heat exchanger disposed in sequence and in fluid communication between each two reactors in series; (w) providing a composition comprising 1230xa; (x) contacting the composition comprising 1230xa with a fluorinating agent such as HF, to produce a product comprising 1233xf; (y) contacting a product comprising 1233xf with a fluorinating agent such as HF, to produce a product comprising 244bb in a liquid or vapor phase reactor; and (z) dehydrochlorinating a product comprising 244bb to produce a product comprising 1234yf in the adiabatic reaction zone.

There is also provided a process for the preparation of 1234yf comprising the following steps: (v′) providing an adiabatic reaction zone comprising at least two serially-connected adiabatic reactors and having a heat exchanger disposed in sequence and in fluid communication between each two reactors in series; (w′) providing a composition comprising 243db; (x′) contacting the composition comprising 243db with a dehydrohalogenating agent or dehydrohalogenating catalyst to produce a product comprising 1233xf; (y′) contacting a product comprising 1233xf with a fluorinating agent such as HF, to produce a product comprising 244bb in a liquid or vapor phase reactor; and (z′) dehydrochlorinating a product comprising 244bb to produce a product comprising 1234yf in the adiabatic reaction zone.

The present disclosure also provides a process for the preparation of 1234yf which may comprise the following steps: (v″) providing an adiabatic reaction zone comprising at least two serially-connected adiabatic reactors and having a heat exchanger disposed in sequence and in fluid communication between each two reactors in series; (w″) providing a composition comprising 243db; (x″) contacting the composition comprising 243db with a dehydrohalogenating agent or dehydrohalogenating catalyst to produce a product comprising 1233xf in the adiabatic reaction zone; (y″) contacting a product comprising 1233xf with a fluorinating agent such as HF, to produce a product comprising 244bb in a liquid or vapor phase reactor; and (z″) dehydrochlorinating a product comprising 244bb to produce a product comprising 1234yf.

The dehydrochlorinating steps (z) and (z′) are performed as disclosed herein, which comprises (aa) introducing a starting material comprising a product comprising 244bb into a first adiabatic reactor of the serially-connected reactors, producing a reaction product; (bb) passing the reaction product from a preceding adiabatic reactor to a heat exchanger, producing an intermediate product; (cc) introducing the intermediate product from the heat exchanger to a subsequent adiabatic reactor, producing a reaction product; (dd) optionally repeating steps (bb) and (cc) in sequence one or more times; and (ee) recovering a final product comprising a haloolefin, wherein the final product is the reaction product produced in a final adiabatic reactor. Optionally step (z″) is also performed as set forth above for steps (z) and (z′).

Similarly, the dehydrochlorinating step (x″) is performed in an adiabatic reaction zone as disclosed herein, which comprises (aa′) introducing a starting material comprising 243db into a first adiabatic reactor of the serially-connected reactors, producing a reaction product; (bb′) passing the reaction product from a preceding adiabatic reactor to a heat exchanger, producing an intermediate product; (cc′) introducing the intermediate product from the heat exchanger to a subsequent adiabatic reactor, producing a reaction product; (dd′) optionally repeating steps (bb′) and (cc′) in sequence one or more times; and (ee′) recovering a final product comprising a haloolefin, wherein the final product is the reaction product produced in a final adiabatic reactor. The haloolefin of step (ee′) comprises 1233xf. Step (x″) is followed by steps (y″) and (z″).

The steps (w)-(y), (w′)-(y′), (w″), (y″) (z″) may be performed using known methods with all of their attendant variations, and such methods are not reproduced here for brevity. For example, in step (z″), 244bb is dehydrochlorinated pyrolytically or catalytically to produce a product comprising the desired product 1234yf as a component of the reactor effluent.

The reaction as set forth in step (z), (z′) and (z″) above may be carried out at a temperature range of from about 200° C. to about 800° C., from about 300° C. to about 600° C., or from about 400° C. to about 500° C. Suitable reactor pressures range from about 0 psig to about 200 psig, from about 10 psig to about 150 psig, or from about 20 to about 100 psig or from about 40 psig to about 80 psig.

The processes set forth in steps (v)-(z), (v′)-(z′) and (v″)-(z″) optionally further comprise treating the product comprising 1233xf produced in steps (x), (x′) and (x″) prior to using the treated product comprising 1233xf in steps (y), (y′) and (y″), respectively. By “treating” is meant herein to separate 1233xf from the product produced in steps (x), (x′) and (x″) and/or purifying 1233xf from the product comprising 1233xf to provide a treated product comprising 1233xf. For purpose of clarity, “a product comprising 1233xf” in step (y), (y′) or (y″) may be the product from step (x), (x′) or (x″), respectively, or the product after treating the product from step (x), (x′) or (x″), respectively, as set forth herein.

The processes set forth in steps (v)-(z), (w′)-(z′) and (v″)-(z″) optionally further comprise treating the product comprising 244bb produced in steps (y), (y′) and (y″) prior to using the treated product comprising 244bb in steps (z), (z′) and (z″), respectively. By “treating” is meant herein to separate 244bb from the product produced in steps (y), (y′) and (y″) and/or purifying 244bb from the product comprising 244bb to provide a treated product comprising 244bb. For purpose of clarity, “a product comprising 244bb” in step (z), (z′) or (z″) may be the product from step (y), (y′) or (y″), respectively, or the product after treating the product from step (y), (y′) or (y″), respectively, as set forth herein.

In process step (x), a composition comprising 1230xa is contacted with a fluorinating agent in the presence of a fluorination catalyst, producing a product mixture comprising 1233xf. In one embodiment, step (x) is performed in the vapor phase, with a fluorination catalyst. The vapor phase fluorination catalyst may be chosen from metal oxides, hydroxides, halides, oxyhalides, inorganic salts thereof and their mixtures any of which may be optionally halogenated, wherein the metal includes, but is not limited to chromium, aluminum, cobalt, manganese, nickel, iron, and combinations of two or more thereof. In another embodiment, step (x) is performed in the liquid phase with a fluorination catalyst. The liquid phase fluorination catalyst may be chosen from metal chlorides and metal fluorides, including, but not limited to, SbCl₅, SbCl₃, SbF₅, SnCl₄, TiCl₄, FeCl₃ and combinations of two or more of these.

In process step (x′) or process step (x″), 243db is dehydrochlorinated to produce a product mixture comprising 1233xf. In this step, dehydrochlorination may be performed in the vapor phase with a dehydrochlorinating catalyst or in the liquid phase with a dehydrochlorinating agent, such as a base. For example, WO 2012/115934 discloses vapor phase reaction of 243db with a carbon catalyst. WO 2012/115938 discloses vapor phase reaction of 243db with a chromium oxyfluoride catalyst. WO 2017/044719 discloses reaction of 243db with a fluorinated alkane in the presence of a fluorination catalyst to produce 1233xf, as well as other compounds useful for producing 1234yf. WO 2017/044724 discloses liquid phase reaction of 243db with caustic. Other methods may be used when starting with a compound having Formula (III) as will be known to those skilled in the art.

Step (x″) is a dehydrochlorination step that may be performed in accordance with the disclosure provided herein in an adiabatic reaction zone.

The process may further comprise one or more steps prior to step (v′) or prior to step (v″). In one embodiment, a process comprises prior to step (v′) or prior to step (v″), steps (t′) and (u′) and steps (t″) and (u″), respectively, is performed which comprises: (t′) or (t″) contacting 250fb with HF and a catalyst under conditions to produce a product comprising 1243zf; and (u′) or (u″) contacting a product comprising 1243zf with chlorine in the presence or absence of a catalyst to produce a product comprising 243db.

The product of step (t′) or (t″) may undergo separation and/or purification prior to using the product in step (u′) or (u″). The product of step (u′) or (u″) may undergo separation and/or purification prior to using the product in step (v′) or (v″). For purpose of clarity, “a product comprising 1243e in step (u′) or (u”) may be the product from step (t′) or (t″), respectively, or the product after treating the product from step (t′) or (t″), respectively, as set forth herein.

Following step (z), step (z′) or step (z″), a process for the preparation of 1234yf may further comprise separation steps to achieve desired degrees of separation of 1234yf from other components present in the product and/or other processing to achieve desired purity. For example, the product from step (z) or step (z′) or step (z″) comprising 1234yf may further comprise one or more of HCl, HF, unconverted 244bb, 3,3,3-trifluoropropyne, 245cb, and 1233xf (the latter of which is mainly carried over from previous step (y) or step (y′) or step (y″), respectively).

HCl may be optionally recovered from the result of a dehydrochlorination reaction. Recovery of HCl may be conducted by conventional distillation where it is removed from the distillate. Alternatively, HCl may be removed or recovered using water or caustic scrubbers. When a water scrubber is used, HCl is removed as an aqueous solution. When a caustic scrubber is used, HCl is removed from the reaction zone as a chloride salt in aqueous solution.

After the recovery or removal of HCl, the remainder of the product from dehydrochlorinating step may be transferred to a distillation column for separation. For example, 1234yf may be collected from the overhead of the column, and optionally, the collected 1234yf may be transferred to another column for further purification. Of the remaining material not collected from the overhead, a fraction may accumulate in a reboiler. For example, this fraction may comprise 1233xf and 244bb. Upon separation from the fraction, 244bb may be returned as a recycle to the dehydrochlorinating step (z) or step (z′) or step (z″).

The present disclosure also provides an adiabatic reaction zone for a dehydrohalogenation process as disclosed herein. There is provided a reaction zone comprising at least two reactors, each reactor operating adiabatically, wherein a heat exchanger is arranged between the at least two reactors.

The adiabatic reaction zone of this disclosure comprises (a) a first adiabatic reactor in fluid communication with a starting material source from which flows a starting material comprising a hydrohaloalkane to the first adiabatic reactor, in which the starting material is converted to a reaction product; (b) a heat exchanger in fluid communication with and downstream from the first adiabatic reactor and through which flows the reaction product, wherein reaction product is heated to provide an intermediate product; (c) a subsequent adiabatic reactor in fluid communication with and downstream from the heat exchanger and through which flows the intermediate product from the heat exchanger, wherein the intermediate product reacts to form a reaction product; and optionally, (d) one or more combinations of a heat exchanger and a subsequent reactor in series, and in fluid communication with the subsequent adiabatic reactor in (c), wherein for each heat exchanger, a reaction product is heated to form an intermediate product, and for each adiabatic reactor, the intermediate product reacts to form a reaction product. Optionally, the adiabatic reaction zone further comprises a heat exchanger upstream of, and in fluid communication with the first adiabatic reactor.

The adiabatic reaction zone may comprise two or more subsequent adiabatic reactors. As set forth above, the adiabatic reaction zone comprises a first adiabatic reactor and a subsequent adiabatic reactor. In one embodiment, the adiabatic reaction zone comprises at least three adiabatic reactors. Thus, such reaction zone comprises a first adiabatic reactor, a second adiabatic reactor, and a third adiabatic reactor, wherein each of the second and third adiabatic reactors is a subsequent adiabatic reactor, the third adiabatic reactor also being the final adiabatic reactor.

A reaction system may comprise the adiabatic reaction zone as disclosed herein and a separation system and/or purification system downstream from and in fluid communication with the adiabatic reaction zone.

The reaction system may comprise operations upstream from and in fluid communication with the adiabatic reaction zone, including means for preheating the starting material. In one embodiment, the reaction system comprises a heat exchanger upstream of and in fluid communication with the reaction zone to preheat the starting material. In one embodiment, the reaction system comprises a vaporizer to vaporize the starting material, which vaporizer is in fluid communication with the adiabatic reaction zone.

### DETAILED DESCRIPTION OF THE FIGURES

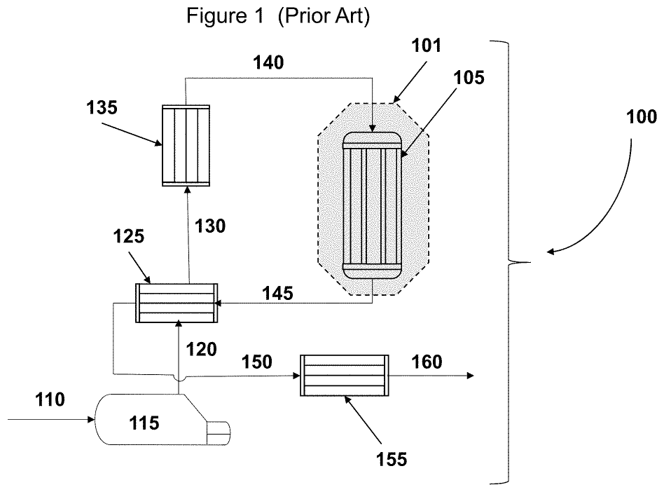

FIG. 1 is a flow diagram illustrating a reaction system of the prior art for a dehydrohalogenation process wherein reaction system 100 has single multi-tubular reactor 105, where multiple tubes are illustrated with the multiple lines within the reactor. Reaction zone 101 consists of reactor 105 and is identified by the shaded area enclosed in dotted lines. Starting material 110, comprising hydrohaloalkane, enters vaporizer 115, in which starting material becomes vaporized starting material 120, which passes through heat exchanger 125, producing heated starting material 130. From heat exchanger 125, heated starting material 130 passes through superheater 135 from which super-heated starting material 140. Super-heated starting material 140 is introduced to multi-tubular reactor 105. From reactor 105, reaction product 145 passes through heat exchanger 125 to provide cooled reaction product 150. Cooled reaction product 150 is then further cooled by passing through heat exchanger 155 to provide product 160.

FIG. 2 is a flow diagram illustrating a dehydrohalogenation process of this disclosure wherein reaction system 200 comprises adiabatic reaction zone 205 consisting of three adiabatic reactors in series. Adiabatic reaction zone 205 consists of adiabatic reactors 260, 261, and 262 as well as heat exchangers 250, 280 and 281 and is identified by the shaded area enclosed in dotted lines (step (a)). Starting material 210, comprising hydrohaloalkane, enters vaporizer 215 in which starting material becomes vaporized starting material 220, which passes through heat exchanger 225, producing heated starting material 230. From heat exchanger 225, heated starting material 230 passes through superheater 235, producing super-heated starting material 240. Super-heated starting material 240 enters adiabatic reaction zone 205 and passes through heat exchanger 250, to produce starting material 251 for first adiabatic reactor 260 (step (a′)). Starting material 251 is introduced to first adiabatic reactor 260 (step (b)). From first adiabatic reactor 260, reaction product 270 passes through heat exchanger 280, in which reaction product 270 is heated and exits as intermediate product 271 (step (c)). Intermediate product 271 is introduced to a subsequent (second) adiabatic reactor 261, in which is produced reaction product 272 (step (d)). From second adiabatic reactor 261, reaction product 272 passes through heat exchanger 281, in which reaction product 272 is heated and exits as intermediate product 273 (step (e), repeating step (c)). Intermediate product 273 is introduced to a subsequent (third and final) adiabatic reactor 262, in which is produced reaction product 274 (step (e), repeating step (d)). From third adiabatic reactor 262, reaction product 274 passes through heat exchanger 225 to provide cooled reaction product 275. Cooled reaction product 275 is then further cooled by passing through heat exchanger 255 to provide product 276, which can be recovered (step (e)).

FIG. 2 illustrates use of process stream—reaction product 274—as heat exchange fluid as heat is exchanged between vaporized starting material 220 and reaction product 274.

### Selected Embodiments

Embodiment (1) provides a process for dehydrohalogenating a hydrohaloalkane in an adiabatic reaction zone, which process comprises: (a) providing an adiabatic reaction zone comprising at least two serially-connected adiabatic reactors and having a heat exchanger disposed in sequence and in fluid communication between each two reactors in series; (b) introducing a starting material comprising a hydrohaloalkane into a first adiabatic reactor of the serially-connected reactors, producing a reaction product; (c) passing the reaction product from a preceding reactor to a heat exchanger, producing an intermediate product; (d) introducing the intermediate product from the heat exchanger to a subsequent adiabatic reactor, producing a reaction product; (e) optionally repeating steps (c) and (d) in sequence one or more times; and (f) recovering a final product comprising a haloolefin, wherein the final product is the reaction product produced in a final adiabatic reactor, which is a subsequent adiabatic reactor having no subsequent adiabatic reactor in the adiabatic reaction zone downstream from the final adiabatic reactor.

Embodiment (2) is the process of Embodiment (1) wherein the hydrohaloalkane has the formula Y¹Y²CH—CXY³Y⁴, where X is F, Cl, Br or I and each of Yⁱ is independently H, F, Cl, Br, or I; an alkyl group or a haloalkyl group, wherein i is 1, 2, 3 and 4 and halo is F, Cl, Br, or I, provided that at least one Yⁱ is not H or at least one Yⁱ is a haloalkyl group.

Embodiment (3) is the process of Embodiment (2) wherein the hydrohaloalkane is a hydrohaloethane.

Embodiment (4) is the process of Embodiment (2) wherein the hydrohaloalkane is 1-chloro-1,1-difluoroethane (CF₂ClCH₃).

Embodiment (5) is the process of Embodiment (2) wherein the hydrohaloalkane is a hydrohalopropane and the haloolefin is a halopropene.

Embodiment (6) is the process of Embodiment (2) wherein the hydrohalopropane is chosen from CF₃CFClCH₃, CF₃CHFCH₂Cl, CF₃CHClCH₂F, CF₃CH₂CHFCl, CF₃CHFCH₂Cl, CF₃CHClCH₃, CF₃CHFCH₂F, CF₃CH₂CF₂H, CF₃CF₂CH₃, CF₃CFClCH₂F, CF₃CHFCHFCl, CF₃CHClCHF₂, CF₃CH₂CF₂Cl, CF₃CHClCH₂Cl, CCl₃CH₂CHCl₂, CF₃CH₂CH₂Cl, CF₃CHClCH₃, CCl₃CHClCH₂Cl, CCl₃CH₂CH₂Cl, CH₂ClCCl₂CHCl₂, and mixtures of two or more thereof.

Embodiment (7) is the process of Embodiment (5) wherein the hydrohalopropane comprises a hydrochlorofluoropropane and the halopropene comprises a hydrofluoropropene.

Embodiment (8) is the process of Embodiment (5) wherein the hydrohalopropane is CF₃CFClCH₃ and the halopropene is CF₃CF═CH₂.

Embodiment (9) is the process of Embodiment (8) further comprising, upstream of step (b), the following steps: (w) providing a composition comprising 1,1,2,3-tetrachloropropene (1230xa); (x) contacting the composition comprising 1230xa with a fluorinating agent such as HF, to produce a product comprising 1233xf; (y) contacting a product comprising 1233xf with a fluorinating agent such as HF, to produce a product comprising 244bb in a liquid or vapor phase reactor; and optionally, (z) separating 244bb from the product of step (y), wherein the product of step (y) or, if optional step (z) is performed, the product of step (z), is the starting material in step (b).

Embodiment (10) is the process of Embodiment (8) further comprising, upstream of step (b), the following steps: (w′) providing a composition comprising CF₃CHClCH₂Cl (243db); (x′) contacting the composition comprising 243db with a dehydrohalogenating agent or dehydrohalogenating catalyst to produce a product comprising CF₃CCl═CH₂ (1233xf); (y′) contacting a product comprising 1233xf with a fluorinating agent such as HF, to produce a product comprising CF₃CFClCH₃ (244bb) in a liquid or vapor phase reactor; and optionally, (z′) separating 244bb from the product of step (y′), wherein the product of step (y′) or, if optional step (z′) is performed, the product of step (z′), is the starting material in step (b).

Embodiment (11) is the process of Embodiment (10) further comprising prior to step (w′): (t′) contacting CCl₃CH₂CH₂Cl (250fb) with HF and a catalyst under conditions to produce a product comprising CF₃CH═CH₂ (1243zf); and (u′) chlorinating a product comprising 1243zf to produce a product comprising CF₃CHClCH₂Cl (243db) by contacting 1243zf with chlorine in the presence or absence of a catalyst.

Embodiment (12) is the process of Embodiment (8) further comprising, upstream of step (b), the following steps: (w″) providing a composition comprising CF₃CHClCH₂Cl (243db); (x″) contacting the composition comprising 243db with a dehydrohalogenating agent or dehydrohalogenating catalyst to produce a product comprising CF₃CCl═CH₂ (1233xf) in the adiabatic reaction zone; (y″) contacting a product comprising 1233xf with a fluorinating agent such as HF, to produce a product comprising CF₃CFClCH₃ (244bb) in a liquid or vapor phase reactor; and optionally, (z″) separating 244bb from the product of step (y″), wherein the product of step (y″) or, if optional step (z″) is performed, the product of step (z″), is the starting material in step (b).

Embodiment (13) is the process of Embodiment (12) further comprising prior to step (w″): (t″) contacting CCl₃CH₂CH₂Cl (250fb) with HF and a catalyst under conditions to produce a product comprising CF₃CH═CH₂ (1243zf); and (u″) chlorinating a product comprising 1243zf to produce a product comprising CF₃CHClCH₂Cl (243db) by contacting 1243zf with chlorine in the presence or absence of a catalyst.

Embodiment (14) is the process of any of Embodiments (9), (10), (11), (12) or (13) further comprising treating the product comprising CF₃CCl═CH₂ (1233xf) to separate 1233xf from the product comprising 1233xf.

Embodiment (15) is the process of any of Embodiments (9), (10), (11), (12) or (13) further comprising treating the product comprising CF₃CFClCH₃ (244bb) to separate 244bb from the product comprising 244bb.

Embodiment (17) is the process of any of Embodiments (9), (10), (11), (12), (13) or (14) further comprising treating the product comprising CF₃CFClCH₃ (244bb) to separate 244bb from the product comprising 244bb.

Embodiment (18) is the process of any of Embodiments (9), (10), (11), (12), (13) or (15) further comprising treating the product comprising CF₃CCl═CH₂ (1233xf) to separate 1233xf from the product comprising 1233xf.

Many aspects and embodiments have been described above and are merely exemplary and not limiting. After reading this specification, skilled artisans appreciate that other aspects and embodiments are possible without departing from the scope of the present disclosure.

### EXAMPLES

### Comparative Example

In this Comparative Example, a single reactor is operated isothermally at a temperature of 480° C. and a pressure of 70 psig. The reactor has multiple empty tubes with a heat transfer fluid flowing through a shell surrounding the reactor to transfer energy consumed by the endothermic reaction. The reactor is made of Inconel 600 to provide corrosion resistance. A continuous flow of 244bb starting material is fed to the reactor. The reaction product is analyzed after 1 hour and the conversion of 244bb to 1234yf is measured as 16.3%, defined as (moles 1234yf produced)/(moles 244bb fed). The productivity of the single isothermal reactor, defined as (rate of 1234yf production)/(total reactor volume), is given the value of 100 for comparison to Examples 1 and 2. The weight of Inconel 600 and the fabrication cost of a commercial scale reactor designed using Aspen In-Plant Cost Estimator™ version 8.8 (available from Aspen Technology, Inc., Newtown, Pa.) are also set at 100 for comparison to Examples 1 and 2.

### Example 1

In this Example 1, an adiabatic reaction zone consists of two reactors of equal volume operating adiabatically in series. The inlet temperature to the first adiabatic reactor is 480° C. and pressure is 70 psig. The adiabatic reactors comprise empty pipes made of Inconel 600. A continuous flow of 244bb starting material is introduced to the first adiabatic reactor at the same feed rate as in the Comparative Example. The reaction product from the first adiabatic reactor is heated in a heat exchanger to 480° C. before entering the second adiabatic reactor. The reaction product from the second adiabatic reactor is analyzed after 1 hour and the conversion of 244bb to 1234yf is measured as 16.3%. The productivity of the two adiabatic reactors in series is 39 compared to the single isothermal reactor. The total weight of Inconel 600 for both reactors is 50% of the weight required in the Comparative Example. The total fabrication cost is 41% of the cost of the single isothermal reactor used in the Comparative Example.

### Example 2

In this Example 2, an adiabatic reaction zone consists of three reactors of equal volume operating adiabatically in series. The inlet temperature to the first adiabatic reactor is 480° C. and pressure is 70 psig. The reactors are the same diameter as used in Example 1 and are made of empty Inconel 600 pipe. A continuous flow of 244bb starting material is introduced to the first adiabatic reactor at the same feed rate as in Comparative Example and Example 1. The reaction products from the first and second adiabatic reactors are heated in heat exchangers to 480° C. before entering the second and third adiabatic reactors, respectively. The reaction product from the third adiabatic reactor is analyzed after 1 hour and the conversion of 244bb to 1234yf is measured as 16.3%. The productivity of the three adiabatic reactors in series is 55 compared to the single isothermal reactor. The total weight of Inconel 600 for both reactors is 35% of the weight required in the Comparative Example. The total fabrication cost is 28% of the cost of the single isothermal reactor used in the Comparative Example.

1. A process for dehydrohalogenating a hydrohaloalkane in an adiabatic reaction zone, which process comprises: (a) providing an adiabatic reaction zone comprising at least two serially-connected adiabatic reactors and having a heat exchanger disposed in sequence and in fluid communication between each two reactors in series; (b) introducing a starting material comprising a hydrohaloalkane into a first adiabatic reactor of the serially-connected reactors, producing a reaction product; (c) passing the reaction product from a preceding reactor to a heat exchanger, producing an intermediate product to achieve a desired conversion; (d) introducing the intermediate product from the heat exchanger to a subsequent adiabatic reactor, producing a reaction product; (e) optionally repeating steps (c) and (d) in sequence one or more times; and (f) recovering a final product comprising a haloolefin, wherein the final product is the reaction product produced in a final adiabatic reactor, which is a subsequent adiabatic reactor having no subsequent adiabatic reactor in the adiabatic reaction zone downstream from the final adiabatic reactor, wherein the hydrohaloalkane has the formula Y¹Y²CH—CXY³Y⁴, where X is F, Cl, Br or I and each of Yⁱ is independently H, F, Cl, Br, or I; an alkyl group or a haloalkyl group, wherein i is 1, 2, 3 and 4 and halo is F, Cl, Br, or I, provided that at least one Yⁱ is not H or at least one Yⁱ is a haloalkyl group.

2-3. (canceled)

4. The process of claim 1 wherein the hydrohaloalkane is a hydrohaloethane.

5. The process of claim 1 wherein the hydrohaloalkane is a hydrohalopropane and the haloolefin is a halopropene, wherein:
the hydrohalopropane is CF₃CFClCH₃ and the halopropene is CF₃CF═CH₂; or
the hydrohalopropane is CF₃CHFCH₂Cl and the halopropene is CF₃CF═CH₂; or
the hydrohalopropane is CF₃CFClCH₃ and the halopropene comprises E- and/or Z—CF₃CH═CHF; or
the hydrohalopropane is CF₃CHFCH₂Cl and the halopropene comprises E- and/or Z—CF₃CH═CHF; or
the hydrohalopropane is CF₃CFClCH₂F and the halopropene comprises E- and/or Z—CF₃CF═CHF; or
the hydrohalopropane is CF₃CHFCHFCl and the halopropene comprises E- and/or Z—CF₃CF═CHF; or
the hydrohalopropane is CF₃CHClCHF₂ and the halopropene is CF₃CH═CF₂; or
the hydrohalopropane is CF₃CHFCHFCl and the halopropene is CF₃CH═CF₂.

6-17. (canceled)

18. The process of claim 1 wherein at least one adiabatic reactor operates as a pyrolysis reactor.

19. (canceled)

20. The process of claim 6 wherein an inert diluent gas is used as a carrier gas for the hydrochlorofluoropropane.

21. The process of claim 6 wherein the process is a dehydrochlorination process and wherein at least one adiabatic reactor operates as a pyrolysis reactor and the pyrolysis reactor is operated at a temperature of from about 300° C. to about 700° C.

22. The process of claim 1 wherein at least one adiabatic reactor operates as an adiabatic catalytic reactor and the adiabatic catalytic reactor is charged with a catalyst.

23. The process of claim 22 wherein the catalyst is chosen from metal halides, metal oxides, halogenated metal oxides, neutral (or zero oxidation state) metal or metal alloy, or carbon in bulk or supported form.

24-31. (canceled)

32. The process of claim 22 wherein the catalytic reactor is operated at a temperature of from about 150° C. to about 550° C. and a suitable reaction pressure may range from about 0 to about 150 psig.

33-36. (canceled)

37. A process for the preparation of 1234yf which comprises the following steps: (v) providing an adiabatic reaction zone comprising at least two serially-connected adiabatic reactors and having a heat exchanger disposed in sequence and in fluid communication between each two reactors in series; (w) providing a composition comprising 1,1,2,3-tetrachloropropene (1230xa); (x) the composition comprising 1230xa with a fluorinating agent such as HF, to produce a product comprising 1233xf; (y) contacting a product comprising 1233xf with a fluorinating agent such as HF, to produce a product comprising 244bb in a liquid or vapor phase reactor; and (z) dehydrochlorinating a product comprising 244bb to produce a product comprising 1234yf in the adiabatic reaction zone.

38. A process for the preparation of 1234yf comprises the following steps: (v′) providing an adiabatic reaction zone comprising at least two serially-connected adiabatic reactors and having a heat exchanger disposed in sequence and in fluid communication between each two reactors in series; (w′) providing a composition comprising 243db; (x′) contacting the composition comprising 243db with a dehydrohalogenating agent or dehydrohalogenating catalyst to produce a product comprising 1233xf; (y′) contacting a product comprising 1233xf with a fluorinating agent such as HF, to produce a product comprising 244bb in a liquid or vapor phase reactor; and (z′) dehydrochlorinating a product comprising 244bb to produce a product comprising 1234yf in the adiabatic reaction zone.

39. The process of claim 38 further comprising prior to step (v′), (t′) contacting 250fb with HF and a catalyst under conditions to produce a product comprising 1243zf; and (u′) chlorinating a product comprising 1243zf to produce a product comprising 243db by contacting 1243zf with chlorine in the presence or absence of a catalyst.

40. A process for the preparation of 1234yf which comprises the following steps: (v″) providing an adiabatic reaction zone comprising at least two serially-connected adiabatic reactors and having a heat exchanger disposed in sequence and in fluid communication between each two reactors in series; (w″) providing a composition comprising 243db; x″) contacting the composition comprising 243db with a dehydrohalogenating agent or dehydrohalogenating catalyst to produce a product comprising 1233xf in the adiabatic reaction zone; (y″) contacting a product comprising 1233xf with a fluorinating agent such as HF, to produce a product comprising 244bb in a liquid or vapor phase reactor; and (z″) dehydrochlorinating a product comprising 244bb to produce a product comprising 1234yf.

41. The process of claim 40 further comprising prior to step (v″), (t″) contacting 250fb with HF and a catalyst under conditions to produce a product comprising 1243zf; and (u″) chlorinating a product comprising 1243zf to produce a product comprising 243db by contacting 1243zf with chlorine in the presence or absence of a catalyst.

42-43. (canceled)

44. A reaction zone comprising (a) a first adiabatic reactor in fluid communication with a starting material source from which flows a starting material comprising a hydrohaloalkane to the first adiabatic reactor, in which the starting material is converted to a reaction product; (b) a heat exchanger in fluid communication with and downstream from the first adiabatic reactor and through which flows the reaction product, wherein reaction product is heated to provide an intermediate product; (c) a subsequent adiabatic reactor in fluid communication with and downstream from the heat exchanger and through which flows the intermediate product from the heat exchanger, wherein the intermediate product reacts to form a reaction product; and optionally, (d) one or more combinations of a heat exchanger and a subsequent reactor in series, and in fluid communication with the subsequent adiabatic reactor in (c), wherein for each heat exchanger, a reaction product is heated to form an intermediate product, and for each adiabatic reactor, the intermediate product reacts to form a reaction product.
